(19) United States
(12) Patent Application Publication
(10) Pub. No.: US 2009/0193568 A1
(43) Pub. Date: Aug. 6, 2009

(54) Diving Mask

(75) Inventor: Chih-Cheng Shiue, Taipei (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: QBAS CO., LTD., Taipei (TW)

(21) Appl. No.: 12/356,102

(22) Filed: Jan. 20, 2009

(30) Foreign Application Priority Data
Feb. 4, 2008 (TW) 97202358
May 26, 2008 (TW) 97209157
Dec. 3, 2008 (TW) 97221643
Jan. 7, 2009 (TW) 98200164

(51) Int. Cl. A61F 9/02

(52) U.S. Cl. 2/443; 2/452

(57) ABSTRACT

A diving mask is provided. The mask comprises a body, at least one lens and at least one elastic frame. The body comprises at least one lens frame. The one lens has a periphery and is disposed correspondingly in the lens frame. The elastic frame is detachably fastened along the periphery of the lens to absorb the impact force and allow the diving mask to float in water.

This application claims priority to Taiwan Patent Applications No. 097,202,358 filed on Feb. 4, 2008, No. 097,209,157 filed on May 26, 2008, No. 097,221,643 filed on Dec. 3, 2008, and No. 098,200,164 filed on Jan. 7, 2009, in which the disclosures of Patent Application No. 098,200,164 are incorporated herein by reference in their entirety.

### CROSS-REFERENCES TO RELATED APPLICATIONS

Not applicable.

### BACKGROUND OF THE INVENTION

1. Field of the Invention

The present invention relates to a diving mask, and particularly, relates to a diving mask capable of absorbing impact.

2. Descriptions of the Related Art

Popular recreational sports, such as swimming, diving and other water sports, not only are a form of exercise but also are a means of relaxation. To make such water sports comfortable, a diving mask must be used to isolate the surrounding water, thereby providing a comfortable environment for the eyes and extending the duration of the activities. Therefore, the diving mask has become an important and indispensable gear for participants of such activities.

Conventional diving masks generally fall into two categories. FIG. 1 depicts a diving mask 1 of the first kind, which is comprised of a body 11, lenses 12, a hard main frame 13, a hard secondary frame 14 and a strap 15. The diving mask 1 is assembled in the following way: the hard main frame 13 is fixed to a lens frame 111 of the body 11, then the lenses 12 are placed into the hard main frame 13, and finally, the hard secondary frame 14 is snap-fitted around a periphery where the lenses 12 and the hard main frame 13 are connected with each other to secure the lenses 12. In this way, the lenses 12 are securely sealed into the lens frame 111 of the body 11. However, this diving mask 1 uses a large number of parts, making the assembly procedure thereof very time-consuming. Moreover, the hard main frame 13 and the hard secondary frame 14 are both made of rigid materials, thereby making the assembly process more difficult and often leading to water leakage due to poor tightness when assembled. Furthermore, when the conventional diving mask 1 drops to the ground or gets impacted, the hard main frame 13, the hard secondary frame 14 and even the lenses 12 tend to crack and cause water leakage.

FIG. 2 depicts a conventional diving mask 2 of the second kind, which comprises a body 21, lenses 22 and a strap 23. By pre-inserting the lenses 22 into a mold for producing the body 21, the lenses 22 will be incorporated within the injection molded body 21, and then the strap 23 is attached to the body 21 to form the diving mask 2. The diving mask 2 of this kind has fewer parts and is easier to assemble. However, because the diving mask 2 is injection-molded, the diving masks thus produced cannot come with colorful and diversified styles as restricted by the molds and are not able to change the appearance. Furthermore, the diving mask of this kind lacks adequate protection measures, and in case it drops to the ground, the lenses 22 thereof are liable to damage and may cause water leakage.

Additionally, if a diving mask of either kind inadvertently falls off the user's head when used in the water, it will sink into the water or drift away with the water flow, making it difficult to retrieve the diving mask.

In view of this, it is highly desirable in the art to provide a diving mask that is easy to assemble, less likely to be damaged, diversified in style and easy to retrieve when dropped in the water.

### SUMMARY OF THE INVENTION

One objective of this invention is to provide a diving mask provided with improved protection against impact, thereby preventing partial damage to the diving mask and consequent water leakage.

Another objective of this invention is to provide a diving mask that may float when dropped into the water, thereby making it easier for retrieval.

Yet a further objective of this invention is to provide a diving mask and a diving mask set, which has a detachable and replaceable elastic frame. This makes the diving masks and diving mask sets of this invention more diversified in style and colorful, which offers more choices for consumers.

The diving mask structure of this invention comprises a body, at least one lens and at least one an elastic frame. The body comprises at least one lens frame. The at least one lens is disposed correspondingly within the at least one lens frame, and then the at least one elastic frame is detachably disposed along the periphery of the lens and the body. The elastic frame has a very low density which makes the overall density of the diving mask smaller than 1 g/cm³. With this configuration, the at least one elastic frame provides the diving mask with a satisfactory impact absorption capability which may avoid cracking of the lens when dropped on the ground or being impacted. Furthermore, because the diving mask has a density lower than that of water, the diving mask is able to float on water, which makes it more convenient for the user to retrieve. Moreover, the diving masks and diving mask sets of this invention further provide various detachable frames of different colors and with balloon-like gloss, so the diving masks are more diversified in appearance to accommodate the various needs of different consumers.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view of a conventional diving mask;

FIG. 2 is a schematic view of another conventional diving mask;

FIG. 3 is an exploded view of a diving mask of the first embodiment of this invention;

FIG. 4 is an assembled view of the diving mask of the first embodiment of this invention;

FIG. 5 is a partial cross-sectional view of the diving mask of the first embodiment of this invention;

FIG. 6 is a schematic view of a filling valve of this invention;

FIG. 7 is an exploded view of a diving mask of the second embodiment of this invention; and

FIG. 8 is a partial cross-sectional view of the diving mask of the second embodiment of this invention.

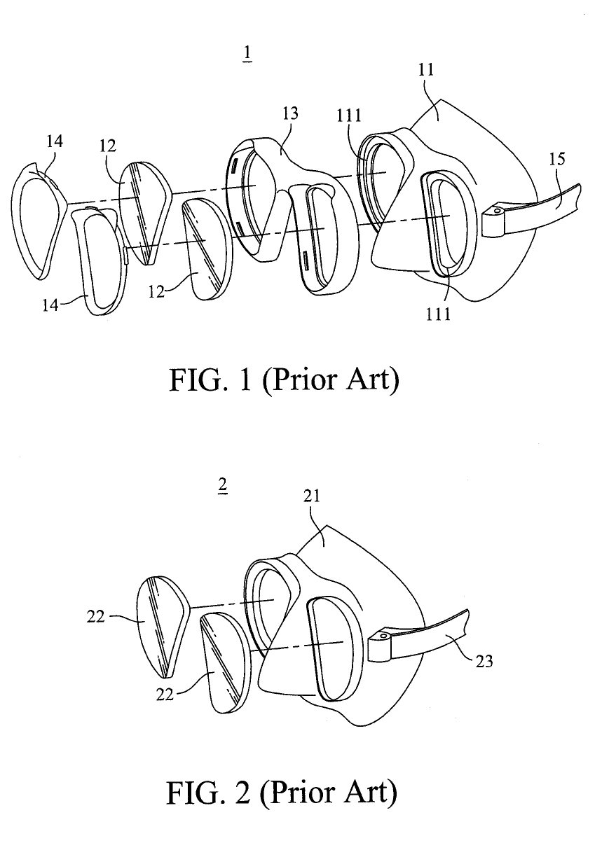

### DESCRIPTION OF THE PREFERRED EMBODIMENT

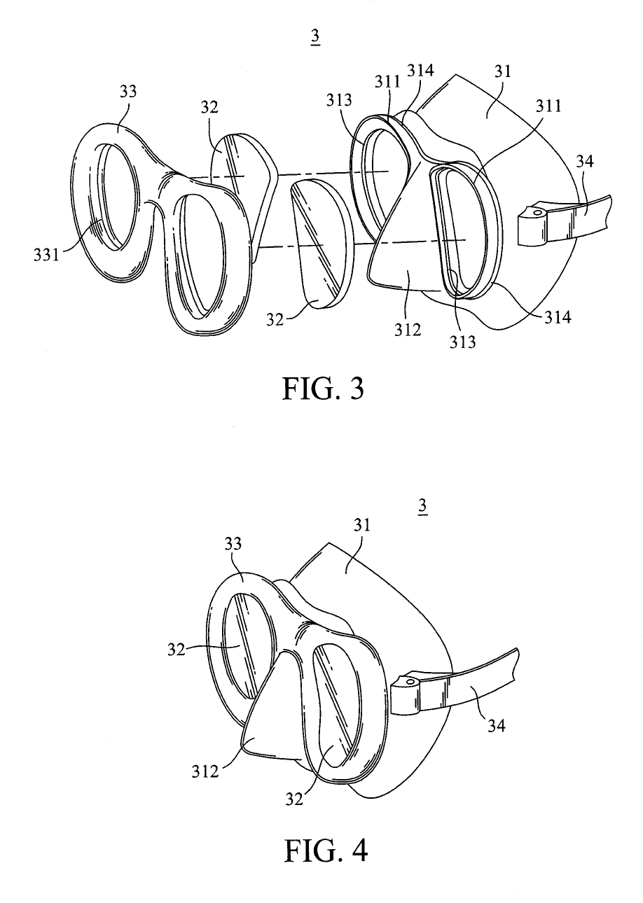

FIGS. 3 and 4 illustrate a diving mask of the first embodiment of this invention. The diving mask 3 comprises a body 31, at least one lens 32 and at least one elastic frame 33. The body 31 comprises at least one lens frame 311. The elastic frame 33 is disposed along the periphery where the lens 32 joins with the lens frame 311. By expanding the elastic frame 33, the elastic frame 33 is adapted to cover the periphery of the lens frame 311 and thus provide a better impact absorption capability.

The body 31, which is preferably made of a soft material such as silicone, is adapted to cover part of the user's face and form an internal space where the ambient liquid is blocked outside. In this embodiment, the body 31 comprises two lens frames 311. Each of the lens frames 311 has a first recess 313 formed at the inner peripheral thereof. Each of the first recesses 313 is adapted to receive and nest one of the two lenses 32 respectively. With a single elastic frame 33 detachably disposed along the peripheries of the two lenses 32, the lenses 32 are securely attached to the body 31. It should be appreciated that the number of elastic frames 33 and that of the lenses 32 of the diving mask 3 in this invention are not only limited to what is described herein. For example, in other examples, the diving mask 3 may be designed with a single elastic frame 33 corresponding to a single unitary lens 32, or with two individual elastic frames corresponding to two lenses 32 disposed in the lens frames 311. Other configurations will readily occur to those of ordinary skill in the art and will not be further described herein.

Preferably, the lenses 32, the body 31 and the lens frame 311 are formed integrally. To this end, the lenses 32 can be insert-molded into the body 31 by placing them into the mold for the body 31 before injection molding, so that the lenses 32 are nested into the first recesses 313 at the same time when the body 31, the lens frame 311 and the first recesses 313 are formed. This may further improve the waterproof capability of the diving mask and obtain the benefit of simplifying the assembly processes in production. Those of ordinary skill in the art may readily know other means for attaching the lenses 32 into the lens frame 311, for example, by adhering, snap-fitting, threading, riveting or pinning, all of which fall within the scope of the claims of this invention. The diving mask 3 of this invention further comprises at least one strap 34 disposed on the body 31 for fastening the diving mask 3 onto the user's head. Also, the body 31 may further have a nose portion 312 accommodated to the user's nose and prevent the ambient liquid from entering into the nose. However, the diving mask 3 may also be formed without the nose portion 312 and adapted to be used as swimming goggles.

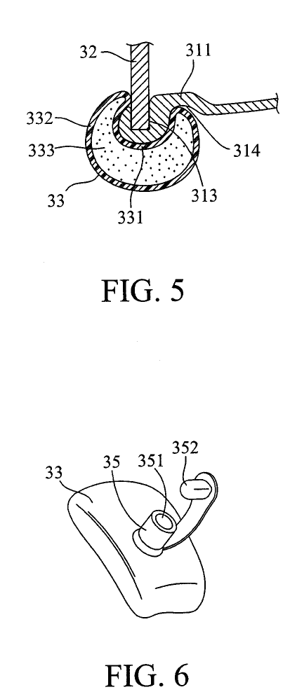

In reference to FIG. 5, the elastic frame 33 has at least one second recess 331 configured to securely receive the lenses 32 and the lens frames 311 and to protect the structure at the periphery of the lens frame 311. In this embodiment, the elastic frame 33 comprises two second recesses 331 corresponding to the two lenses 32 respectively, which are adapted to be detachably fastened with the two lens frames 311. In this embodiment, the body 31 has a third recess 314 to nest at the edge of the elastic frame 33 so that the elastic frame 33 can be joined to the body 31 more tightly. The user may also assemble or replace the elastic frames 33 with different colors, designs and styles by himself or herself, thus adding value to the diving mask 3 of this invention.

For the dropped diving mask 3 to float on water for easy retrieval, the elastic frame 33 has a density at least less than 1 g/cm³, and preferably ranging from 0.3 g/cm³ to 0.93 g/cm³. With the adequate density of the elastic frame 33, the diving mask 3 should have an average density of less than 1 g/cm³ and is able to float on water. More specifically, any density of the diving mask 3 less than that of the liquid in which the user is diving falls within scope of this invention. The density of 1 g/cm³ set forth above only illustrates a case where the liquid is pure water.

As shown in FIG. 5, the elastic frame 33 of the diving mask 3 of this invention is preferably a hollow structure. The elastic frame 33 comprises an expandable element 332 which is adapted to be infilled with a filler 333. By filling the filler 333 into the expandable element 332 to expand the expandable element 332, the lenses 32 and the body 31 may be joined together more securely and a better cushioning capability is provided. Thus, in case the diving mask 3 is dropped or impacted, the elastic frame 33 can effectively protect the lenses 32 to avoid cracking of the lenses 32 and consequent water leakage. Meanwhile, the expandable element 332 filled with the filler 333 exhibits a balloon-like gloss which, in conjunction with the vivid colors and diversified designs, may prompt stronger purchase desires of children or even adults. Furthermore, the expandable element 332 filled with the filler 333 lowers the density of the elastic frame 33 and the diving mask 3, so that the diving mask 3, when dropping into water, will float on water without sinking to the bottom, thus making it easier for the user to retrieve the mask.

As shown in FIG. 6, the elastic frame 33 of this invention preferably comprises a filling valve 35 disposed on the expandable element 332 of the elastic frame 33. The filling valve 35 comprises a filling spout 351 and a plug 352. The plug 352 is adapted to be plugged into the filling spout 351 to prevent leakage of the filler 333. With the filling valve 35, the user may infill with the filler 333 by blowing air or by using a pressurizing equipment, and may also discharge the filler 333 via the filling valve 35. The filling valve 35 may also be of other filling valve configurations, but not solely limited to what is described above.

The elastic frame 33 may also be a solid structure instead of a hollow structure, and may also absorb impact and provide flotation so that the material thereof has a sufficiently low density and an appropriate elasticity. Hence, the material of the elastic frame 33 may be selected from a group consisting of foam material, rubber, ethylene-vinyl acetate (EVA), polyvinyl chloride (PVC), low density polyethylene (LDPE), poly amide (PA), polyethylene (PE), thermoplastic polyurethane (TPU) or other elastic polymers. These materials also have appropriate deformability, and are adapted for use as materials of the expandable element 332 which expands after being infilled with the filler 333. The material of the filler 333 may be selected from the group consisting of gas, liquid, foam material and the combination thereof, which adapted to be infilled into the expandable element 332.

The elastic frame 33 does not need to be detachably fastened with the body 31 by means of the second recess 331,and may also be adhered, snap-fitted, threaded, riveted, pinned or insert-molded into the body 31, or be permanently fastened to the body 31 by other means. As long as the elastic frame 33 has vivid colors, a low density and appropriate elasticity, it will achieve the purpose of this invention to float and protect the diving mask 3 against damage and consequent water leakage, thereby promoting customers to purchase.

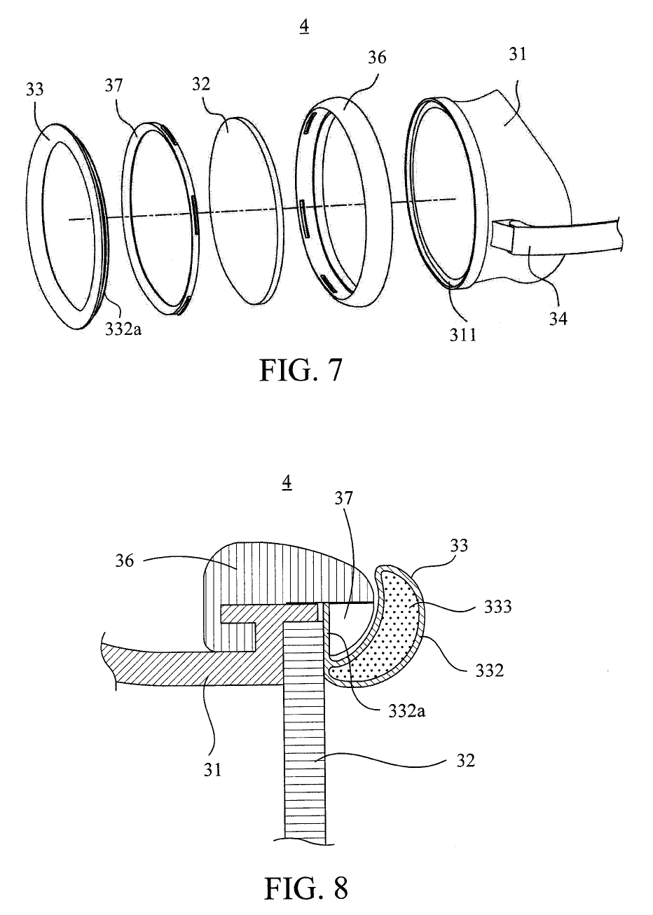

FIGS. 7 and 8 illustrate the second embodiment of the diving mask of this invention. The diving mask 4 of the second embodiment differs from the diving mask 3 of the first embodiment only in that a securing structure similar to that of the conventional diving mask 1 is utilized to secure the elastic frame 33 to the body 31 in the diving mask 4. In this embodiment, the diving mask 4 further comprises at least one first securing frame 36 and at least one second securing frame 37, while the at least one elastic frame 33 is formed with a lug portion 332a. The first step of the assembly of the diving mask 4 is disposing the first securing frame 36 along the periphery of the lens frame 311 of the body 31, and then inserting the lens 32 into the lens frame 311. After the second securing frame 37 is fitted between the elastic frame 33 and the lug portion 332a thereof, the second securing frame 37 and the elastic frame 33 are disposed into the first securing frame 36 together, so that the second securing frame 37 is detachably snap-fitted to the first securing frame 36 and disposed along the periphery of the lens 32. After the completion of the assembly, the lug portion 332a of the elastic frame 33 is clamped between the second securing frame 37 and the lens 32, so as to secure the elastic frame 33 onto the body 31. This will prevent the damage of lens 32, the first securing frame 36 and the second securing frame 37 while falling down or crashing, and therefore overcome the disadvantage of the conventional diving mask 1 that the hard main frame 13 and the hard secondary frame 14 are vulnerable to the damage caused by crashing.

The elastic frame 33 of the diving mask 4 of the second embodiment may utilize the expandable element 332 as that in the first embodiment. By filling the filler 333 into the interior space of the expandable element 332 to expand the expandable element 332, a good cushioning capability is provided. The expandable element 332 also lowers the density of the elastic frame 33 and the diving mask 4, so that the diving mask 4 is adapted to float on the water for easy retrieving while dropped into the water, instead of sinking into the water and causing inconvenience of retrieving. The elastic frame 33 of the second embodiment may be also a solid structure instead of a hollow structure, and be made of the same materials as that of the first embodiment. Specifically, the elastic frame 33 in the second embodiment falls within the scope of this invention as long as the elastic frame 33 renders the diving mask 4 with an overall density smaller than that of the liquid for diving in.

In application, this invention may further provide a diving mask set comprising a series of different elastic frames 33 assembled with a diving mask 3 for replacing with different elastic frames 33. Accordingly, depending on his or her preferences, the user may choose elastic frames of different colors, designs and styles to meet the requirements for appearance.

In summary, when compared to conventional diving marks, the elastic frames of this invention endows the diving mask with a cushioning capability to absorb the impact force and floating capability for the user to easily retrieve the diving mask when accidentally dropped into water. Moreover, it is convenient for the user to replace the elastic frames with different colors, designs and styles.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A diving mask, comprising:
a body comprising at least one lens frame;
at least one lens having a periphery and disposed correspondingly within the at least one lens frame; and
at least one elastic frame detachably disposed along the periphery of the at least one lens.

2. The diving mask as claimed in claim 1, wherein the at least one elastic frame has a density less than 1 g/cm³ and the diving mask has an average density less than 1 g/cm³.

3. The diving mask as claimed in claim 2, wherein the density of the at least one elastic frame ranges from 0.3 g/cm³ to 0.93 g/cm³.

4. The diving mask as claimed in claim 3, wherein the at least one elastic frame comprises an expandable element, adapted to be infilled with a filler.

5. The diving mask as claimed in claim 4, wherein the at least one elastic frame further comprises a filling valve, and the filler is adapted to infill the expandable element through the filling valve.

6. The diving mask as claimed in claim 4, wherein the filler is selected from the group consisting of gas, liquid, foam material and the combination thereof.

7. The diving mask as claimed in claim 3, wherein the at least one elastic frame is made of polymer material.

8. The diving mask as claimed in claim 7, wherein the polymer material is selected from the group consisting of foam material, rubber, ethylene-vinyl acetate (EVA), polyvinyl chloride (PVC), low density polyethylene (LDPE), poly amide (PA), polyethylene (PE), thermoplastic polyurethane (TPU) and the combination thereof.

9. The diving mask as claimed in claim 1, wherein the at least one lens frame has a first recess adapted to receive the at least one lens.

10. The diving mask as claimed in claim 9, wherein the at least one elastic frame has at least one second recess adapted to be detachably fastened with the at least one lens frame.

11. The diving mask as claimed in claim 9, wherein the at least one lens is insert molded within the body and the at least one lens frame.

12. The diving mask as claimed in claim 1, wherein the diving mask comprises two lenses and one elastic frame, the body comprises two lens frames, the lenses are disposed correspondingly in the lens frames, and the elastic frame is in integral detachably disposed along the two peripheries of the lenses.

13. The diving mask as claimed in claim 1, further comprising at least one first securing frame and at least one second securing frame, wherein the at least one first securing frame is detachably disposed along a periphery of the at least one lens frame, the at least one second securing frame is detachably snap-fitted to the at least one first securing frame and disposed along the periphery of the at least one lens, the at least one elastic frame is formed with a lug portion, and the lug portion is clamped between the at least one second securing frame and the at least one lens.

14. The diving mask as claimed in claim 1, further comprising a strap disposed on the body.

15. A diving mask, comprising:
a body comprising at least one lens frame;
at least one lens having a periphery and disposed correspondingly within the at least one lens frame; and
at least one elastic frame fastened along the periphery of the at least one lens;
wherein the elastic frame has a density less than 1 g/cm³ and the diving mask has an average density less than 1 g/cm³.

16. The diving mask as claimed in claim 15, wherein the density of the elastic frame ranges from 0.3 g/cm³ to 0.93 g/cm³.

17. The diving mask as claimed in claim 16, wherein the elastic frame comprises an expandable element, adapted to be infilled with a filler.

18. The diving mask as claimed in claim 17, wherein the elastic frame further comprises a filling valve, and the filler is adapted to infill the expandable element through the filling valve.

19. The diving mask as claimed in claim 17, wherein the filler is selected from the group consisting of gas, liquid, foam material and the combination thereof.

20. The diving mask as claimed in claim 16, wherein the elastic frame is made of polymer material.

21. The diving mask as claimed in claim 20, wherein the polymer material is selected from the group consisting of foam material, rubber, ethylene-vinyl acetate, polyvinyl chloride, low density polyethylene, poly amide, polyethylene, thermoplastic polyurethane and the combination thereof.

22. The diving mask as claimed in claim 15, further comprising a strap disposed on the body.

23. A diving mask set, comprising:
a body comprising at least one lens frame;
at least one lens having a periphery and disposed correspondingly within the at least one lens frame; and
a plurality of elastic frames, wherein each of the elastic frames is detachably disposed along the periphery of the at least one lens.
